(19) United States
(12) Patent Application Publication
(10) Pub. No.: US 2025/0366758 A1
(43) Pub. Date: Dec. 4, 2025

(54) GUIDE APPARATUS, METHOD FOR MANUFACTURING SAME AND METHOD FOR USING SAME

(71) Applicant: SHANGHAI STAIRMED TECHNOLOGY CO., LTD., Shanghai (CN)

(72) Inventors: Zhengtuo ZHAO, Shanghai (CN); Xue LI; Yu BAO, Shanghai (CN)

(21) Appl. No.: 18/876,298

(22) PCT Filed: Jun. 29, 2022
(86) PCT No.: PCT/CN2022/102317

§ 371 Date: Dec. 18, 2024

(30) Foreign Application Priority Data
Jun. 20, 2022 (CN) 202210697141.8

(51) Int. Cl. A61B 5/293; A61B 5/00; A61L 31/02; C25F 3/02

(52) U.S. Cl. CPC A61B 5/293; A61B 5/6851; A61L 31/022; C25F 3/02; A61B 2560/063; A61B 2562/028; A61B 2562/125

(57) ABSTRACT

A guide apparatus (1) for implanting a wire electrode (2) with a through hole provided thereon. The guide apparatus (1) includes, in a length direction thereof: a first portion (111) at an end, a maximum outer diameter size of the first portion (111) being smaller than a size of the through hole; and a second portion (112) connected to the first portion (111), a minimum outer diameter size of the second portion (112) being larger than the size of the through hole, wherein the second portion (112) and the first portion (111) are connected via a step structure.

### TECHNICAL FIELD

The present disclosure relates to a guide apparatus, a method for manufacturing a guide apparatus, a method for guiding a wire electrode, a method for using a guide apparatus, and a method for guiding a wire electrode using a guide apparatus.

### BACKGROUND

Electrophysiological recording by implanted neural electrodes is essential in neuroscience and brings unique prospects for human neurorestoratology. Despite achieving great success and potential, traditional micromachined silicon probes have obvious mechanical mismatches with the neural tissue host, resulting in short-term and long-term interface instability. Extensive efforts have been made to reduce the size and mechanical stiffness of neural probes, so as to improve biocompatibility and recording reliability. However, there is an inherent conflict in the requirements for the hardness of the probes between the minimal invasiveness and the easy insertion into the brain with the minimal surgical damage. In order to eliminate chronic tissue response and reduce the stiffness of the neural probe, it is necessary to make the deformation force of the probe comparable to the cellular force in the neural tissue.

Therefore, flexible electrodes have been developed. However, due to the flexible electrode's own “flexible” characteristic, its implantation becomes a difficult problem. Since the flexible electrode has small sizes in its width direction and thickness direction, and has a size in its length direction that is much larger than those in its width direction and thickness direction. Therefore, in the present disclosure, the flexible electrode is called “wire electrode” according to its shape characteristic.

### SUMMARY

The purpose of the present disclosure is to provide a guide apparatus, a method for manufacturing a guide apparatus, a method for guiding a wire electrode, a method for using a guide apparatus, and a method for guiding a wire electrode using a guide apparatus. The guide apparatus, the method for manufacturing a guide apparatus, the method for guiding a wire electrode, the method for using a guide apparatus, and the method of guiding a wire electrode using a guide apparatus can overcome at least one of the defects in the prior art.

According to a first aspect of the present disclosure, there is provided a guide apparatus for implanting a wire electrode with a through hole provided thereon, the guide apparatus including, in a length direction thereof: a first portion at an end, a maximum outer diameter size of the first portion being smaller than a size of the through hole; and a second portion adjacent to the first portion, a minimum outer diameter size of the second portion being larger than the size of the through hole, wherein the second portion and the first portion are connected via a step structure.

According to a second aspect of the present disclosure, a guide apparatus for a wire electrode is provided, the guide apparatus being constructed to be able to pass through an engagement part of the wire electrode so as to be engaged with the engagement part in order to guide the wire electrode, the guide apparatus having an implantation section constructed to at least partially enter a target object, wherein the implantation section has a first portion at a front end, a second portion adjoining a rear of the first portion, and a transition portion for connecting the first portion and the second portion, wherein the first portion is constructed to be able to pass through the engagement part, and the second portion is constructed to be unable to pass through the engagement part, so that the engagement part of the wire electrode can be stopped at the transition portion.

According to a third aspect of the present disclosure, a method for manufacturing a guide apparatus is provided, which includes: immersing a first section of an etchable blank into an etchant, and performing a first electrochemical etching on the first section, so as to obtain a semi-finished product with one step structure; and immersing a first sub-section at an end of the first section into the etchant, and performing a second electrochemical etching on the first sub-section, so as to obtain the guide apparatus with two step structures, wherein an implantation section of the guide apparatus is obtained on the first section, the implantation section having a first portion corresponding to the first sub-section and a second portion corresponding to the rest of the first section except for the first sub-section, the first portion being thinner than the second portion.

According to a fourth aspect of the present disclosure, a method for manufacturing a guide apparatus is provided, which includes: immersing a first sub-section at an end of a first section of an etchable blank into an etchant, and performing a first electrochemical etching on the first sub-section, so as to obtain a semi-finished product with one step structure; and immersing the first section into the etchant, and performing a second electrochemical etching on the first section, so as to obtain the guide apparatus with two step structures, wherein an implantation section of the guide apparatus is obtained on the first section, the implantation section having a first portion corresponding to the first sub-section and a second portion corresponding to the rest of the first section except for the first sub-section, the first portion being thinner than the second portion.

According to a fifth aspect of the present disclosure, a method for guiding a wire electrode is provided, which includes: placing, at a first position, the wire electrode with a through hole provided at an end portion thereof; aligning a guide apparatus with the through hole, wherein the guide apparatus includes, in a length direction thereof, a first portion at a front end and a second portion adjacent to the first portion, a maximum outer diameter size of the first portion is smaller than a size of the through hole, and a minimum outer diameter size of the second portion is larger than the size of the through hole, wherein the second portion and the first portion are connected via a step structure; moving the guide apparatus forward so that the first portion passes through the through hole and the end portion of the wire electrode is stopped at the step structure due to pressure from the step structure; and moving the guide apparatus to guide at least the end portion of the wire electrode from the first position to a second position.

According to a sixth aspect of the present disclosure, a method for using a guide apparatus is provided, which includes: bringing the guide apparatus close to an engagement part of a wire electrode, wherein the guide apparatus includes a thinner first portion at a front end, a thicker second portion adjoining a rear of the first portion, and a transition portion for connecting the first portion and the second portion, wherein the first portion is constructed to be able to pass through the engagement part, and the second portion is constructed to be unable to pass through the engagement part; passing the first portion through the engagement part of the wire electrode, and stopping the engagement part of the wire electrode at the transition portion; and guiding the wire electrode using the guide apparatus.

According to a seventh aspect of the present disclosure, a method for guiding a wire electrode using a guide apparatus is provided, the guide apparatus including a thinner first portion at a front end, a thicker second portion adjoining a rear of the first portion, and a transition portion for connecting the first portion and the second portion, wherein the first portion is constructed to be able to pass through an engagement part, the second portion is constructed to be unable to pass through the engagement part, a first end portion of the wire electrode is constructed with the engagement part, and the method includes: passing the first portion through the engagement part, and stopping the engagement part of the wire electrode at the transition portion; further moving the guide apparatus to apply pull force to the first end portion of the wire electrode, so as to at least partially separate the wire electrode from a fixation apparatus for the wire electrode; guiding the wire electrode to a target position using the guide apparatus.

Other features of the present disclosure and advantages thereof will become apparent from the following detailed description of exemplary embodiments of the present disclosure with reference to the accompanying drawings.

### BRIEF DESCRIPTION OF THE DRAWINGS

The accompanying drawings, which constitute a part of this specification, illustrate embodiments of the present disclosure and, together with the description, serve to explain the principles of the present disclosure.

FIG. 1A is a schematic diagram of a guide apparatus according to an embodiment of the present disclosure.

FIG. 1B is a schematic diagram of a portion of an example wire electrode that can be used in conjunction with a guide apparatus according to an embodiment of the present disclosure.

FIG. 1C to FIG. 1F are schematic diagrams of an engagement of an example wire electrode with a guide apparatus according to an embodiment of the present disclosure.

FIG. 1G is a schematic diagram of an engagement of an example wire electrode with a guide apparatus according to an embodiment of the present disclosure.

FIG. 1H is a partially enlarged diagram of a guide apparatus according to an embodiment of the present disclosure.

FIG. 1I and FIG. 1J are schematic diagrams of an engagement of an example wire electrode with a guide apparatus according to an embodiment of the present disclosure.

FIG. 2 to FIG. 6 are schematic diagrams of a process of an embodiment of a method for guiding a wire electrode according to an embodiment of the present disclosure.

FIG. 7 to FIG. 10 are schematic diagrams of a process of another embodiment of a method for guiding a wire electrode according to an embodiment of the present disclosure.

FIG. 11A to FIG. 11C are schematic diagrams at steps of a process in which a method for manufacturing a guide apparatus according to an embodiment of the present disclosure is implemented.

FIG. 11D is a schematic diagram at a step of a process in which a method for manufacturing a guide apparatus according to an embodiment of the present disclosure is implemented.

FIG. 11E and FIG. 11F are schematic diagrams of using isolation material in implementing a method for manufacturing a guide apparatus according to an embodiment of the present disclosure.

FIG. 12A is a schematic diagram of a conventional etchant solution etching performed on a piece being etched.

FIG. 12B is a schematic diagram of a method for manufacturing a guide apparatus according to an embodiment of the present disclosure.

FIG. 12C to FIG. 12E are schematic diagrams of making an isolation material form a flat surface in implementing a method for manufacturing a guide apparatus according to an embodiment of the present disclosure.

FIG. 13A and FIG. 13B are schematic diagrams of at least a portion of a guide apparatus according to an embodiment of the present disclosure.

Note that in the embodiments described below, the same reference numeral is sometimes used in common among different drawings to denote the same part or parts having the same function, and a repetitive description thereof will be omitted. In some cases, similar items are indicated using similar reference numbers and letters, and thus, once a certain item is defined in one drawing, it need not be discussed further in subsequent drawings. For ease of understanding, positions, dimensions, ranges,

and the like of the structures shown in the drawings and the like sometimes do not necessarily indicate actual positions, dimensions, ranges, and the like. Therefore, the present disclosure is not limited to the positions, dimensions, ranges, and the like disclosed in the drawings and the like.

### DETAILED DESCRIPTION

The present disclosure will be described below with reference to the accompanying drawings, which illustrate several embodiments of the present disclosure. It should be understood, however, that the present disclosure may be presented in many different ways and is not limited to the embodiments described below; in fact, the embodiments described below are intended to make the disclosure of the present disclosure more complete, and to fully convey the scope of protection of the present disclosure to those skilled in the art. It should also be understood that the embodiments disclosed herein can be combined in various ways to provide more additional embodiments.

It should be understood that the terminology used herein is only for describing specific embodiments, and is not intended to limit the scope of the present disclosure. All terms (including technical and scientific terms) used herein have meanings commonly understood by those skilled in the art, unless otherwise defined. Well-known functions or structures may not be described in detail for brevity and/or clarity.

Herein, when it is described that an element is located “on”, “attached” to, “connected” to, “coupled” to, or “contacted” with another element, and so on, the element can be directly located on, attached to, connected to, coupled to, or contacted with the other element, or an intermediate element can be present.

In contrast, when it is described that an element is “directly located on”, “directly attached to”, “directly connected to”, “directly coupled to”, or “directly contacted with” another element, no intermediate element will be present. Herein, a feature arranged “adjacent” to another feature, can refer to the feature having a portion overlapped with the adjacent feature or a portion located above or below the adjacent feature.

Herein, elements or nodes or features “coupled” together may be mentioned. Unless expressly stated otherwise, “couple” means that an element/node/feature may be mechanically, electrically, logically, or otherwise connected to another element/node/feature directly or indirectly to allow interaction, even though the two features may not be directly connected. That is, “couple” is intended to include both direct and indirect connections of elements or other features, including a connection using one or more intermediate elements.

Herein, a spatial relationship term, such as “above”, “below”, “left”, “right”, “front”, “back”, “upper”, or “lower”, may describe a relationship between a feature and another feature in a drawing. It should be understood that the spatial relationship term includes different orientations of a device in use or operation in addition to an orientation shown in the drawing. For example, when the device in the drawing is turned upside down, a feature originally described as “below” another feature may now be described as “above” the other feature at this time. The device may also be otherwise oriented (rotated 90 degrees or at other orientations), and at this time, a relative spatial relationship will be interpreted accordingly.

Herein, the term “A or B” includes “A and B” and “A or B”, rather than exclusively including only “A” or including only “B”, unless specifically stated otherwise.

Herein, the term “exemplary” means “serving as an example, instance, or illustration”, and not as a “model” that is to be reproduced exactly. Any implementation exemplarily described herein is not necessarily to be construed as preferred or advantageous over other implementations. Moreover, the present disclosure is not limited by any expressed or implied theory presented in the above TECHNICAL FIELD, BACKGROUND, SUMMARY, or DETAILED DESCRIPTION.

Herein, the term “substantially” means encompassing any minor variations caused by imperfections in design or manufacturing, tolerances of devices or components, environmental effects and/or other factors. The term “substantially” also allows for differences from a perfect or ideal situation caused by parasitic effect, noise, and other practical considerations that may exist in a practical implementation.

In addition, for reference purposes only, similar terms such as “first” and “second” can also be used herein, and thus are not intended to be limiting. For example, unless clearly indicated by the context, the terms “first”, “second” and other such numerical terms involving structures or elements do not imply a sequence or order.

It should be further understood that the term “comprise/include”, when used herein, specifies the presence of stated features, steps, operations, units and/or components, but do not preclude the presence or addition of one or more other features, steps, operations, units and/or components, and/or combinations thereof.

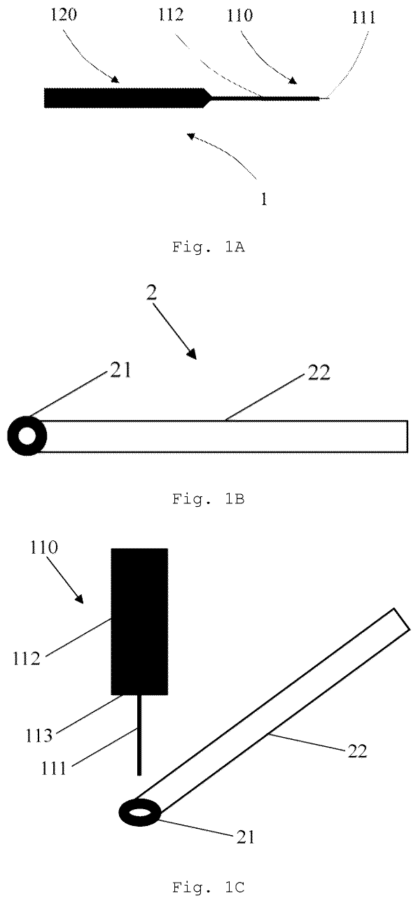

FIG. 1A is a schematic diagram of a guide apparatus 1 according to the present disclosure. FIG. 1B is a schematic diagram of a portion of an exemplary wire electrode 2 that can be used in conjunction with the guide apparatus 1 according to an embodiment of the present disclosure, which shows an engagement part 21 at a front end of the wire electrode 2 and a section 22 adjacent to the engagement part 21 of the wire electrode 2 in a plan view. FIG. 1C to FIG. 1F are schematic diagrams of an exemplary engagement of the guide apparatus 1 with the example wire electrode 2 according to an embodiment of the present disclosure. Wherein, FIG. 1C and FIG. 1D show the example in a perspective view, and FIG. 1E and FIG. 1F show the example in a side view. For simplicity, FIG. 1C to FIG. 1F show only a portion of an implantation section 110 of the guide apparatus 1 in FIG. 1A that is near a front end.

As shown in the figures, the guide apparatus 1 for implanting the wire electrode 2 according to the present disclosure includes the implantation section 110 that at least partially enters a target object when the wire electrode 2 is implanted. The implantation section 110 includes, in the length direction thereof: a first portion 111 at an end or say a front end; and a second portion 112 adjoining or connected to a rear of the first portion 111. There may be a transition portion 113 for connecting the first portion 111 and the second portion 112 between the first portion 111 and the second portion 112, so that the first portion 111 and the second portion 112 are connected via the transition portion 113. In the guide apparatus 1 according to various embodiments of the present disclosure, the transition portion 113 has a step structure.

The guide apparatus 1 may be constructed to be able to pass through the engagement part 21 of the wire electrode 2 so as to be engaged with the engagement part 21 in order to guide the wire electrode 2. FIG. 1B shows a specific example of the wire electrode 2. In this example, the engagement part 21 of the wire electrode 2 is constructed as a through hole. A maximum radial size of the first portion 111 may be smaller than a size of the through hole, and a minimum radial size of the second portion 112 may be larger than the size of the through hole. In other words, the first portion 111 may be constructed to be able to pass through the through hole, and the second portion 112 may be constructed to be unable to pass through the through hole. Thus, when the guide apparatus 1 is aligned with the through hole of the wire electrode 2 and moves in a longitudinal direction towards the through hole, that is, moves downwards in the view of FIG. 1C and FIG. 1D, the engagement part 21 of the wire electrode 2 that has the through hole can be stopped at the transition portion 113 after the first portion 111 of the guide apparatus 1 passes through the through hole, as shown in FIG. 1E. In addition, when the engagement part 21 of the wire electrode 2 is stopped at the transition portion 113, a section (which may refer to a section 221 in FIG. 1G, FIG. 1I and FIG. 1J) of the wire electrode 2 that is adjacent to the engagement part 21 is attached to the transition portion 113.

Shapes of the first portion 111 and the second portion 112 may be substantially columnar. In an embodiment, the shapes of the first portion 111 and the second portion 112 are substantially cylindrical. In other embodiments, the shape of the first portion 111 and/or the second portion 112 may also be other shape, for example, polygon prism, such as triangular prism, quadrilateral prism or pentagonal prism or the like. In more embodiments, the shape of the first portion 111 and/or the second portion 112 may also be substantially conical, frustum-shaped, etc. Since an axial size of the first portion 111 and/or the second portion 112 is much larger than the radial size, the first portion 111 and/or the second portion 112 are depicted as columnar in some drawings of the present disclosure. An outer diameter of the first portion 111 or the second portion 112 mentioned in the present disclosure refers to a diameter of a circle when a cross-section of the columnar portion is substantially circular, or refers to a diameter of a circumcircle of a polygon when the cross-section of the columnar portion is polygonal. The outer diameter size of the first portion 111 may be between 5 μm and 15 μm, preferably between 7 μm and 8 μm, and the outer diameter size of the second portion 112 may be between 40 μm and 70 μm, preferably between 40 μm and 50 um. A length of the first portion 111 may be between 0.05 mm and 0.3 mm, preferably between 0.2 mm and 0.3 mm. A total length of the first portion 111 and the second portion 112 is not less than 3 mm, preferably between 3 mm and 4 mm. The first portion 111 and the second portion 112 may be formed integrally. In some embodiments, the first portion 111 and the second portion 112 may be integrally formed of tungsten or stainless steel by an etching process. In other words, the implantation section 110 may be formed of tungsten or stainless steel by the etching process. In some embodiments, the implantation section 110 may be made of a metal, alloy, carbon fiber or diamond with a Young's modulus greater than 20 GPa.

In some embodiments, the transition portion 113 with the step structure is constructed to be platform-like, as shown in

FIG. 1C and FIG. 1G. In these embodiments, the platform-like transition portion 113 has a plane substantially perpendicular to a longitudinal direction of the guide apparatus 1, for example, a lower surface of the transition portion 113 in FIG. 1C and FIG. 1G. It should be illustrated that, unless particularly emphasized on smooth or even surfaces, when referring to “plane” in the present disclosure, it is not to emphasize that it is a smooth surface, but it's intended to illustrate that an extension direction thereof can be substantially regarded as a plane. For example, in the example shown in FIG. 1H, the lower surface of the transition portion 113 is also referred to as a “plane” in the present disclosure, although it has protrusions and depressions. A size of the lower surface of the transition portion 113 is greater than the size of the through hole of the engagement part 21, which enables the engagement part 21 to be stopped at the lower surface of the transition portion 113 after the first portion 111 of the guide apparatus 1 passes through the engagement part 21 of the wire electrode 2, due to a pressure from the lower surface of the transition portion 113 in the longitudinal direction of the guide apparatus 1. In addition, when the engagement part 21 is stopped at the lower surface of the transition portion 113, the section 221 of the wire electrode 2 that is adjacent to the engagement part 21 is attached to the transition portion 113. In some embodiments, the transition portion 113 with the step structure is constructed to be slope-like, as shown in FIG. 1I and FIG. 1J. In these embodiments, the slope-like transition portion 113 may not have a plane perpendicular to the longitudinal direction of the guide apparatus 1, but is constructed to have an increasing outer diameter in a direction from the first portion 111 to the second portion 112. In other words, in these embodiments, the transition portion 113 is constructed as a tapered portion. Since the outer diameter size of the first portion 111 is significantly smaller than the outer diameter size of the second portion 112, and an axial size of the transition portion 113 is small (i.e., a length of the transition portion 113 extending in the longitudinal direction of the guide apparatus 1 is limited), the slope of the transition portion 113 has a small gradient, in other words, it's a relatively gentle slope. For example, in the example shown in FIG. 1I, a section of the transition portion 113 that is close to the second portion 112 has a relatively gentle slope. It should be illustrated that the gradient here refers to a degree of obliqueness of a straight line or a tangent to a curve of the slope with respect to an abscissa axis (a coordinate axis perpendicular to the longitudinal direction of the guide apparatus 1). Such construction enables an outer peripheral edge at a specific position of the transition portion 113 to apply pressure in the longitudinal direction of the guide apparatus 1 to the engagement part 21 of the wire electrode 2, so that the engagement part 21 can be stopped at the specific position of the transition portion 113, and the section 221 of the wire electrode 2 that is adjacent to the engagement part 21 is attached to the transition portion 113.

By making the outer diameter size of the first portion 111 of the guide apparatus 1 smaller than the size of the through hole and making the outer diameter size of the second portion 112 larger than the size of the through hole, on the one hand, when using the guide apparatus 1 to implant the wire electrode 2, at the through hole, the wire electrode 2 can be stopped at the transition portion 113 for the first portion 111 and the second portion 112 without positional shift in the longitudinal direction of the guide apparatus 1, so as to achieve a more accurate and repeatable positioning of the implantation position.

On the other hand, a guide apparatus having a progressive head end portion (for example, a tapered head end portion with a gradually varying size) is known in the prior art, and a portion of the progressive head end portion thereof can pass through a through hole of a wire electrode to guide the wire electrode. When the progressive head end portion guides the wire electrode to enter a target object, the wire electrode slides from a thinner section of the progressive head end portion to a thicker section due to the resistance that the wire electrode bears in the implantation process, resulting in the through hole of the wire electrode tightly stuck on the progressive head end portion due to the friction between the head end portion and the through hole. Therefore, when the progressive head end portion is removed, there is a risk that the wire electrode is undesirably displaced or brought out of the target object. And the head end portion of the guide apparatus 1 according to the embodiments of the present disclosure has the step structure. After the first portion 111 of the guide apparatus 1 passes through the through hole of the engagement part 21, the engagement part 21 can be stopped at the step structure due to the pressure from the step structure in the longitudinal direction of the guide apparatus 1, rather than being locked on the guide apparatus 1 tightly due to the friction between the engagement part 21 and the guide apparatus 1. Therefore, when the guide apparatus 1 is retracted from the implanted target object, the wire electrode 2 will not be brought out of the implanted target object nor will the position of the positioned wire electrode 2 be shifted again.

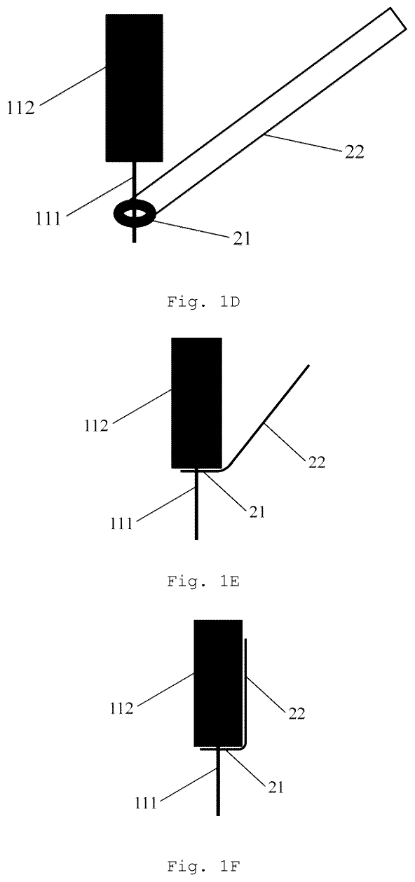

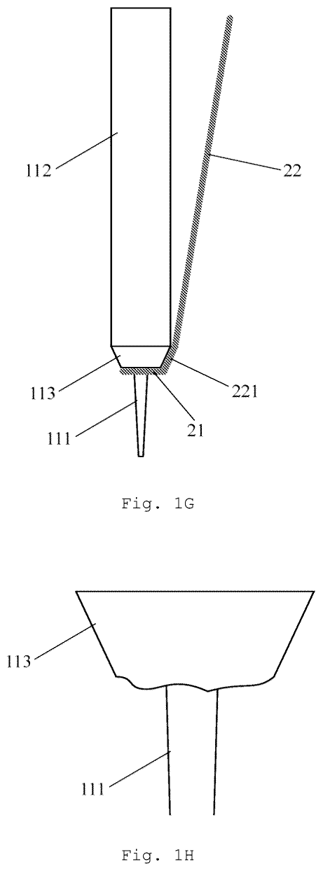

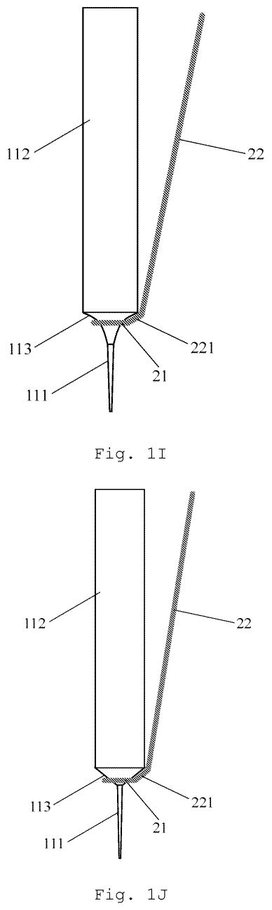

In addition to the implantation section 110, the guide apparatus 1 may also include a fixation section 120, preferably columnar, that fixes the guide apparatus 1 to a movement apparatus (not shown) for implantation, wherein the fixation section 120 may be fixed in a receiving tube 4 for the guide apparatus as shown in FIG. 2 to FIG. 6. The fixation section 120 may be adjacent to the implantation section 110, especially the second portion 112, wherein the fixation section 120 may be formed integrally with the implantation section 110, or they can be connected in a manner of material locking, such as welding or adhering, to be connected in one piece. In some embodiments, the fixation section 120 may also be constructed separately from the implantation section 110 and they are connected in a manner of force fit or shape fit. In the case of the force-fit connection, the fixation section 120 may clamp the implantation section 110. The fixation section 120 may be constructed to be hollow, and the second portion 112 of the implantation section 110 can be received and fixed (for example, clamped) in the hollow structure of the fixation section 120. An outer diameter size of the fixation section 120 may be greater than the outer diameter size of the second portion 112. The outer diameter size of the fixation section 120 may be between 100 μm and 200 μm.

In the case where the fixation section 120 may also be constructed separately from the implantation section 110, the fixation section 120 and the implantation section 110 may be formed of different materials. For example, the fixation section 120 may be made of stainless steel, and the implantation section 110 may be made of tungsten. Therefore, a more flexible choice of materials can be achieved. A stiffness of the material of the fixation section 120 may be higher than a stiffness of the material of the implantation section 110. Thus, compared with the implantation section 110, the fixation section 120, in addition to a greater structural stiffness due to the larger outer diameter size, also has a greater material stiffness than that of the implantation section 110. Therefore, deformation is less likely to occur, ensuring stiffness requirement required when fixed on the movement apparatus and supporting the implantation section 110. For the implantation section 110, in the case where the size of the implantation section 110 adopts the size defined in the present disclosure and the material of the implantation section 110 is tungsten, on the one hand, it is possible to ensure that the implantation section 110 has appropriate stiffness during the implantation, and the appropriate stiffness can support the penetration force when passing through a surface of a tissue to be implanted. On the other hand, the implantation section 110 has a small radial size and a large axial size, that is, the implantation section 110 as a whole is elongate needle-like. Therefore, during implantation, even if undesirable deformation occurs, the implantation section 110 will not easily break to partially remain in the brain. In addition, the material of the implantation section 110 may also be stainless steel. Since the stiffness of stainless steel is smaller than that of tungsten, in order to achieve mechanical strength comparable to that of the tungsten-made implantation section 110 for penetrating the surface of the target object to be implanted, the implantation section 110 made of stainless steel needs to be thicker.

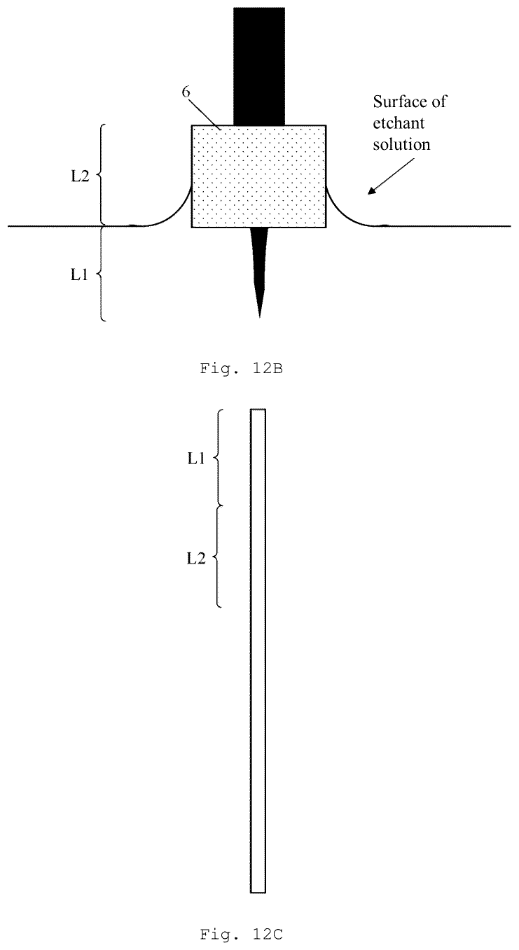

An embodiment of a method for manufacturing the guide apparatus 1 according to the present disclosure is described below in conjunction with FIG. 11A to FIG. 11F. First, a blank is cut to have an appropriate length of 1.5 cm to 2.0 cm, as shown in FIG. 11A, and can be mounted on a fixation stage using copper tape and conductive adhesive such as silver adhesive. Then a first section (for example, a right section of the blank shown in FIG. 11A, a section L in FIG. 11F) of the etchable blank can be immersed into an etchant, and a first electrochemical etching is performed on the first section, so as to obtain a semi-finished product with one step structure as shown in FIG. 11B. The etchant may be an alkaline solution, for example, a potassium hydroxide solution. The first electrochemical etching can be performed at a voltage between 20 V and 40 V, especially 29 V, wherein the voltage is applied to the blank through graphite. A length of the immersed first section corresponds to a length of the finally obtained implantation section 110, i.e., not less than 3 mm, for example, 3 mm to 4 mm. After a first target diameter is reached, the first electrochemical etching can be stopped. The first target diameter may correspond to the outer diameter of the second portion 112 of the implantation section 110. Next, a first sub-section (for example, a right end portion of the semi-finished product as shown in FIG. 11B, a section L1 in FIG. 11F) at an end of the first section can be immersed into the etchant, and a second electrochemical etching is performed on the first sub-section, so as to obtain the guide apparatus 1 with two step structures as shown in FIG. 11C. A length of the first sub-section corresponds to a length of the first portion 111 of the implantation section 110, for example, 0.05 mm to 0.3 mm.

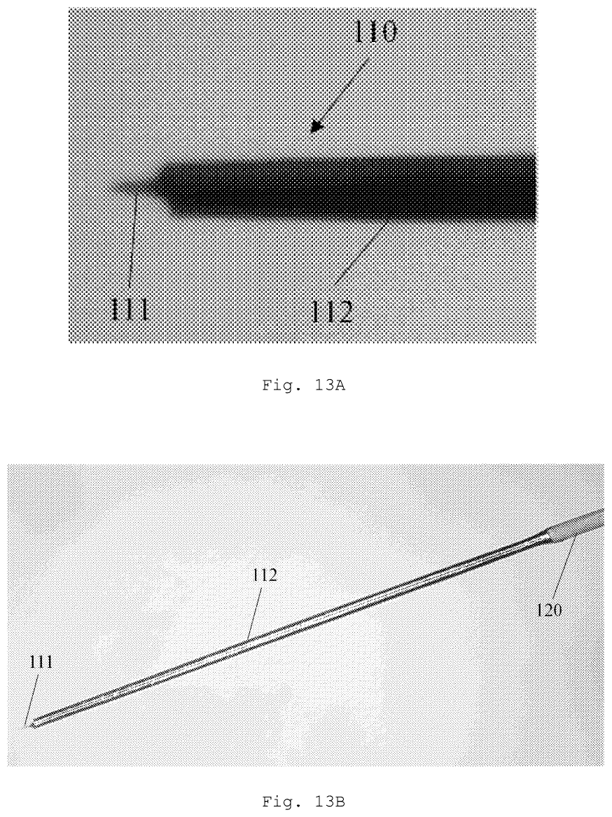

Before the second electrochemical etching is performed, the first sub-section and a portion at the rear of the blank that is connected with the conductive adhesive can be kept exposed, and a second sub-section of the first section that does not require the electrochemical etching and optionally the rest of the blank are covered with an isolation material 6, so that during the second electrochemical etching, only the first sub-section in the first section is in contact with the etchant. Alternatively, referring to FIG. 11E and FIG. 11F, it is possible to cover only the second sub-section L2 of the first section L that adjoins the first sub-section L1 with the isolation material 6, and at least a portion of the second sub-section L2 and the first sub-section L1 are immersed in the etchant solution, so that the second sub-section L2 is not in contact with the etchant during the second electrochemical etching. The isolation material 6 may be a silicone adhesive. After the silicone adhesive is dried, the second electrochemical etching is performed on the first sub-section with a voltage between 0.5 V and 5 V, especially 2 V, to achieve a final target diameter. The final target diameter may correspond to the outer diameter of the first portion 111 of the implantation section 110. Finally, the implantation section 110 of the guide apparatus 1 is obtained on the first section, and the implantation section 110 has the first portion 111 corresponding to the first sub-section and a second portion 112 corresponding to the rest of the first section except for the first sub-section. A schematic diagram of the implantation section 110 of the exemplary guide apparatus 1 may be shown in FIG. 13A and FIG. 13B.

In the above embodiment, the second portion 112 of the implantation section 110 is first formed, and then the first portion 111 of the implantation section 110 is formed. In another embodiment, a semi-finished product with the first portion 111 of the implantation section 110 may also be formed first, and then the entire implantation section 110 (i.e., the first portion 111 and the second portion 112) may be formed. Here, the first sub-section (for example, the right end portion of the blank as shown in FIG. 11A) at the end of the first section of the etchable blank may be immersed into an etchant first, and a first electrochemical etching is performed on the first sub-section, so as to obtain a semi-finished product with one step structure as shown in FIG. 11D. A length of the immersed first sub-section corresponds to a length of the first portion 111 of the finally obtained implantation section 110, for example, 0.05 mm to 0.3 mm. After a difference between the diameter of the etched portion and the diameter of the blank reaches a target value, the first electrochemical etching can be stopped. The target value may correspond to a difference of the outer diameter of the second portion 112 and the outer diameter of the first portion 111 of the implantation section 110. Then, the first section (for example, the right section of the semi-finished product as shown in FIG. 11D) can be immersed into the etchant, and a second electrochemical etching is performed on the first section, so as to obtain the guide apparatus 1 with two step structures as shown in FIG. 11C. Wherein, the first electrochemical etching can be performed at a lower voltage, and the second electrochemical etching can be performed at a higher voltage.

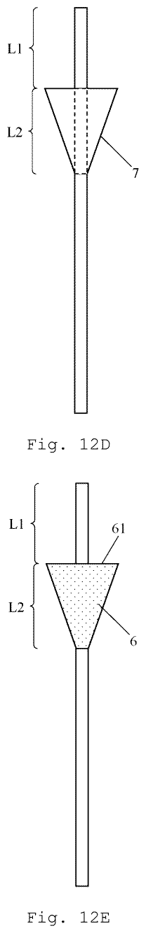

As described above, the transition portion 113 of the guide apparatus 1 according to various embodiments of the present disclosure has a step structure which is constructed to be platform-like with a plane substantially perpendicular to the longitudinal direction of the guide apparatus 1, or to be slope-like with a small gradient. Therefore, in the above-mentioned method for manufacturing the guide apparatus, it is desirable to be able to etch the step structure as flat as possible. However, due to a surface tension of liquid, as shown in FIG. 12A, a surface of the etchant solution will arch at the etched piece immersed therein, and will attach to an outer wall of the etched piece at a position that is higher than a horizontal surface of the solution, making it difficult to form the etched piece with the step structure having the required gentle slope. For example, only the progressive head end portion as shown in FIG. 12A can be obtained. Therefore, as shown in FIG. 12B, it is necessary to make an end of the isolation material 6 that is close to the first sub-section L1 form a substantially flat surface, i.e., the lower surface of the isolation material 6 in the view shown in FIG. 12B is a substantially flat surface. In addition, it is also necessary that this substantially flat surface is as perpendicular to a longitudinal direction of the etched piece as possible. For example, an angle (refer to an angle x in FIG. 11F) between the substantially flat surface and a longitudinal direction of the second sub-section L2 is not less than a threshold angle of, for example, 60 degrees.

FIG. 12C to FIG. 12E show a method that can make the isolation material 6 form the above-described substantially flat surface. The blank to be etched is placed upright and the first sub-section L1 is positioned above the second sub-section L2, as shown in FIG. 12C. A container 7 for holding the isolation material is provided around the outside of the second sub-section L2, and an opening of the container 7 faces upward, as shown in FIG. 12D. In the specific example, the container 7 is conical. Those skilled in the art should understand that in other embodiments, the container 7 may have any shape that has an upward opening and may be provided around the outside of the blank to hold the isolation material. The flowable isolation material 6 is poured into the container 7, and an upper surface 61 of the flowable isolation material 6 will form a substantially flat surface under the effect of gravity, as shown in FIG. 12E. It should be illustrated that in the example shown in FIG. 12C to FIG. 12E, the step structure is not shown on the blank to be etched. Those skilled in the art should understand that the example in the drawings is for simplicity only. The blank to be etched in FIG. 12C to FIG. 12E may be in the state as shown in FIG. 11A, or it can also be in the state as shown in FIG. 11B.

In some embodiments, the first section L1 of the blank may be vertically immersed into the etchant. For example, after the blank as shown in FIG. 12E is turned upside down, the first section L1 (possibly together with at least a portion of the second section L2 that is close to the first section L1) is vertically immersed into the etchant. Wherein, an etching state of the first section of the blank can be observed laterally with an observation apparatus. In some embodiments, the first section L1 of the blank may also be immersed laterally (e.g., obliquely, or transversely) into the etchant, making it possible to observe an electrochemical etching e of f the first section L1 from above with the observation apparatus for observing the electrochemical etching state of the first section L1. The above obliquely refers to that an angle between the longitudinal direction of the blank and the surface of the etchant solution is less than 90 degrees. The above transversely refers to that the longitudinal direction of the blank is substantially parallel to the surface of the etchant solution. For example, it is immersed into the etchant from a side wall of a container holding the etchant. The observation apparatus may observe the electrochemical etching state of the first section L1 vertically from above. The observation apparatus may be constructed as an optical microscope. Therefore, in the embodiment where the first section L1 of the blank is laterally immersed into the etchant, there is no need to rely on a lateral microscope, but a universal microscope with an observation direction from above can be used.

The blanks can be electrochemically etched separately and individually. In some embodiments, electrochemical etching can be performed on and the multiple blanks simultaneously, electrochemical etching process of each blank is separately controlled, respectively. Herein, before performing the first electrochemical etching, each blank may be connected in series with a separate switching element respectively, and corresponding a corresponding blank and a sub-circuits each including corresponding switching element are connected in parallel to a main circuit. Specifically, each blank is connected in series with one independent switch, and then all the series circuits are connected in parallel to the main circuit. Next, electrochemical etching is performed on the first sections of the blanks. At this time, observation is performed with the microscope, and a circuit corresponding to a blank whose first section has completed the electrochemical etching process is turned off (at this moment, the blank is still immersed in the etchant, but no current will flow therethrough as the circuit is turned off, so no further erosion will occur). Meanwhile, other blanks will continue to be etched until all of the blanks have completed the etching process. Then, all the guide apparatuses 1 finally obtained will be taken out together.

FIG. 2 to FIG. 6 are schematic diagrams of a process of an embodiment of a method for guiding the wire electrode 2 according to an embodiment of the present disclosure. In the embodiment of FIG. 2 to FIG. 6, the fixation section 120 of the guide apparatus 1 can be received and fixed in the receiving tube 4 for the guide apparatus, while the implantation section 110 is at least partially exposed for guiding the wire electrode 2.

As shown in FIG. 2 to FIG. 4, the wire electrode 2 is first placed at a first position, with a through hole provided at an end portion of the wire electrode 2 (which may refer to FIG. 1B), and the wire electrode 2 is adhered to a substrate 3 located at the first position. The guide apparatus 1 is then aligned with the through hole, wherein, in FIG. 2 to FIG. 6, the guide apparatus 1 is shown as the implantation section 110 and the receiving tube 4 for the guide apparatus. Then the guide apparatus 1 is moved forward, or in other words, the guide apparatus 1 is brought close to the through hole of the wire electrode 2, so that the first portion 111 of the implantation section 110 (which may refer to FIG. 1D) passes through the through hole and the end portion of the wire electrode 2 is stopped at the transition portion 113 for the first portion 111 and the second portion 112 (which may refer to FIG. 1E). Next, the guide apparatus 1 is moved to guide at least the end portion of the wire electrode 2 from the first position to a second position. Wherein, during the guiding of at least the end portion of the wire electrode 2 from the first position to the second position, the wire electrode 2 is at least partially peeled off from the substrate 3.

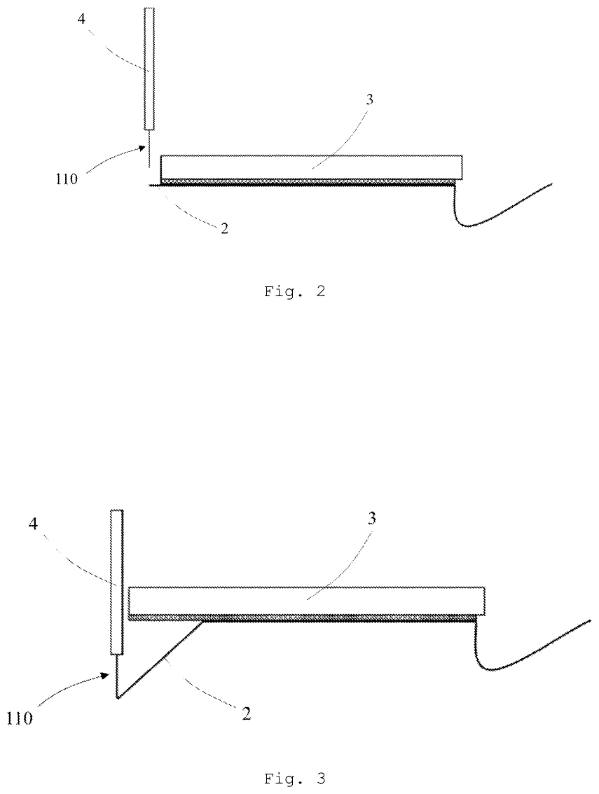

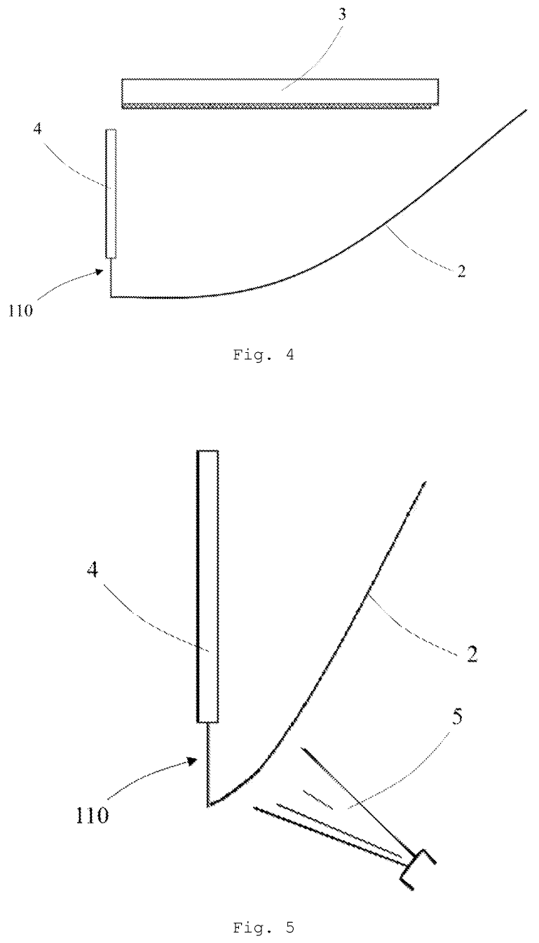

As shown in FIG. 5 and FIG. 6, a liquid 5 may be sprayed on the wire electrode 2 before using the guide apparatus 1 to guide the wire electrode 2 so that at least the first section of the wire electrode 2 is attached to the transition portion 113 and/or the second portion 112 of the guide apparatus 1. The schematic diagram after the attachment may refer to FIG. 1F. Thus, the wire electrode 2 may be fixed to the transition portion and/or the second portion 112 without additional holding apparatus. To this end, the transition portion 113 and/or the second portion 112 may be constructed to have a smooth surface for facilitating the attachment of the first section of the wire electrode 2.

In the embodiment shown in FIG. 2 to FIG. 6, in order to peel off the wire electrode 2, the guide apparatus 1 can be moved perpendicularly to, i.e., at an angle of 90 degrees with respect to, a fixation plane for the wire electrode 2 on the fixation apparatus, in a direction away from the fixation plane, in order to apply pull force to the first end portion of the wire electrode 2.

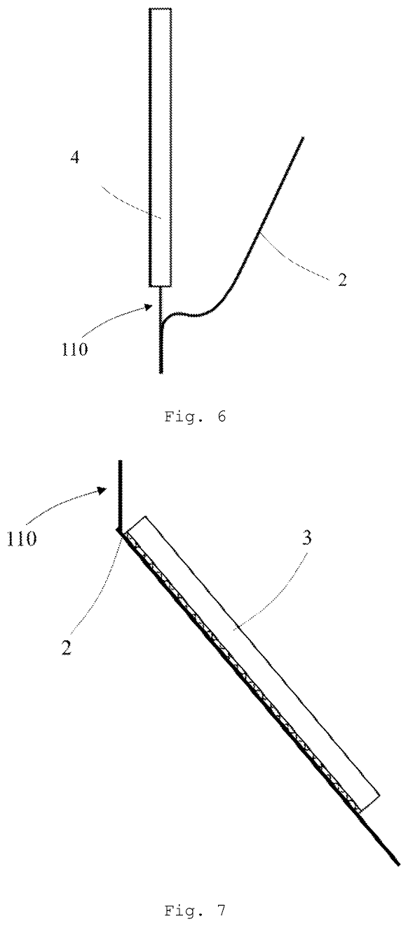

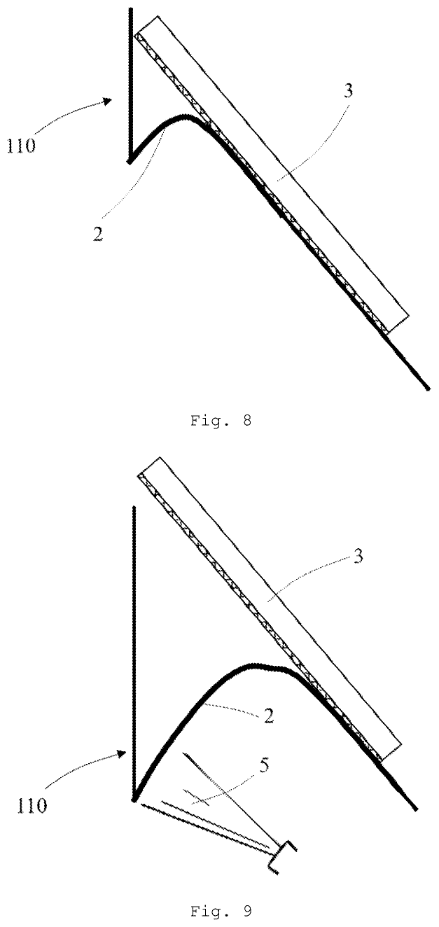

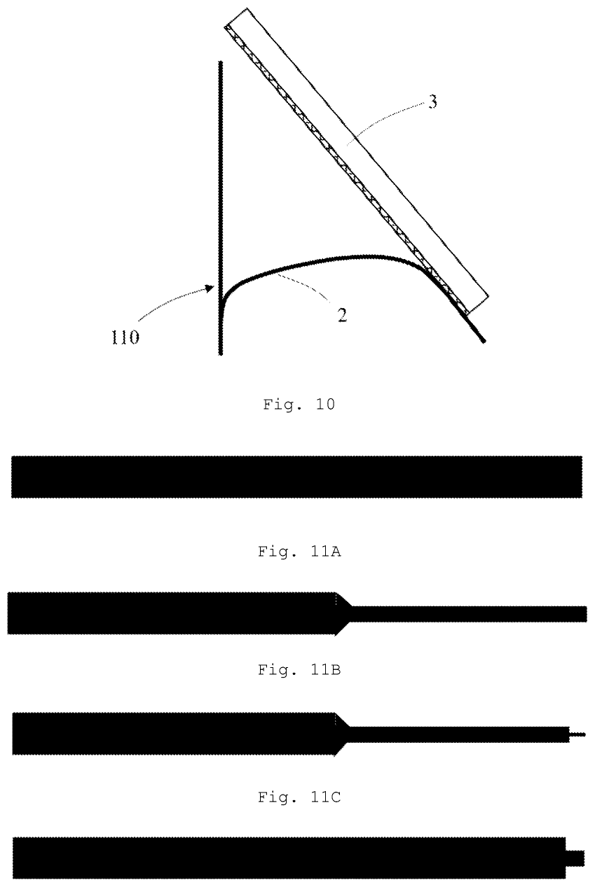

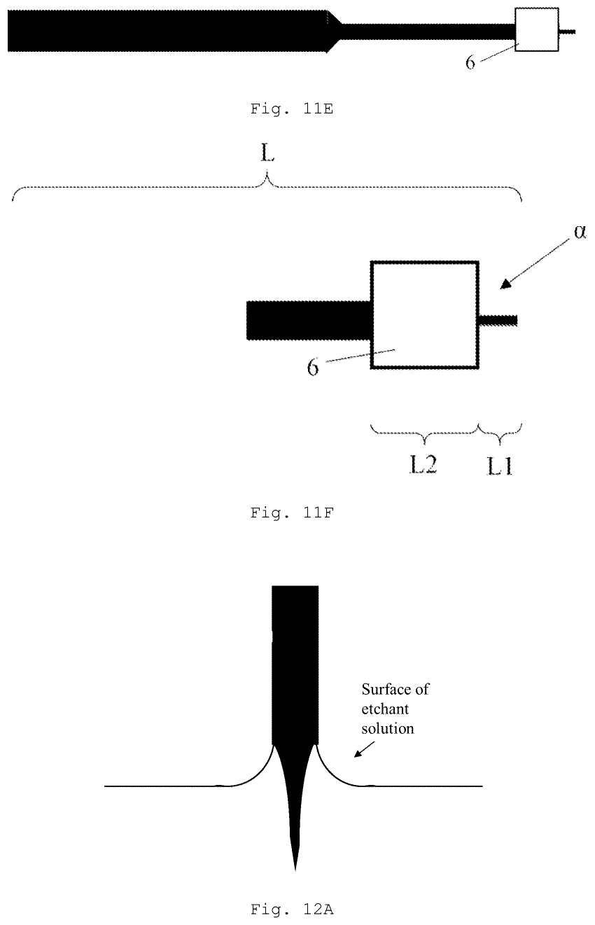

FIG. 7 to FIG. 10 are schematic diagrams of a process of another embodiment of a method for guiding the wire electrode 2 according to an embodiment of the present disclosure. A difference between the embodiment shown in FIG. 7 to FIG. 10 and the embodiment shown in FIG. 2 to FIG. 6 lies in that, in the embodiment shown in FIG. 7 to FIG. 10, the fixation plane on the fixation apparatus forms a non-right angle, for example, a 60-degree angle, with respect to a guide direction of the guide apparatus 1. In order to avoid duplication of description, other aspects of the embodiment shown in FIG. 7 to FIG. 10 can refer to the description of FIG. 2 to FIG. 6. Those skilled in the art should understand that in other embodiments, the fixation plane on the fixation apparatus may also form other angle than 60 degrees or 90 degrees with respect to the guide direction of the guide apparatus 1, as long as the guide apparatus is moved at an angle from the fixation plane on the fixation apparatus where the wire electrode is present, in order to apply pull force to the end portion of the wire electrode.

Although some specific embodiments of the present disclosure have been described in detail by examples, those skilled in the art will understand that the foregoing examples are merely used for illustration, but not for limiting the scope of the present disclosure. The embodiments disclosed herein may be combined arbitrarily without departing from the spirit and scope of the present disclosure. Those skilled in the art will further understand that various modifications may be made to the embodiments without departing from the scope and spirit of the present disclosure. The scope of the present disclosure is defined by the appended claims.

What is claimed is:

1. A guide apparatus for implanting a wire electrode with a through hole provided thereon, the guide apparatus comprising, in a length direction thereof:
a first portion at an end, a maximum outer diameter size of the first portion being smaller than a size of the through hole; and
a second portion connected to the first portion, a minimum outer diameter size of the second portion being larger than the size of the through hole,
wherein the second portion and the first portion are connected via a step structure.

2. The guide apparatus of claim 1, wherein the step structure is constructed to be platform-like and/or slope-like.

3. The guide apparatus of claim 1, wherein the outer diameter size of the first portion is between 5 μm and 15 μm.

4. The guide apparatus of claim 1, wherein the outer diameter size of the first portion is between 7 μm and 8 μm.

5. The guide apparatus of claim 1, wherein the outer diameter size of the second portion is between 40 μm and 70 μm.

6. The guide apparatus of claim 1, wherein the outer diameter size of the second portion is between 40 μm and 50 μm.

7. The guide apparatus of any one of claims 1 to 6, wherein a length of the first portion is between 0.05 mm and 0.3 mm, and a total length of the first portion and the second portion is not less than 3 mm.

8. The guide apparatus of any one of claims 1 to 6, wherein the first portion and the second portion are formed integrally.

9. The guide apparatus of any one of claims 1 to 6, wherein the first portion and the second portion are formed integrally of substantially columnar tungsten or stainless steel by an etching process.

10. The guide apparatus of any one of claims 1 to 6, further comprising a substantially columnar fixation section for fixing the guide apparatus to a movement apparatus for implantation, the fixation section being connected with the second portion and an outer diameter size of the fixation section being larger than the outer diameter size of the second portion.

11. The guide apparatus of claim 10, wherein the outer diameter size of the fixation section is between 100 μm and 200 μm.

12. The guide apparatus of claim 10, wherein a total length of the guide apparatus is between 1.5 cm and 2.0 cm.

13. The guide apparatus of claim 10, wherein the guide apparatus is formed integrally.

14. The guide apparatus of claim 10, wherein the guide apparatus is formed integrally of substantially columnar tungsten or stainless steel by an etching process.

15. A guide apparatus for a wire electrode, the guide apparatus being constructed to be able to pass through an engagement part of the wire electrode so as to be engaged with the engagement part in order to guide the wire electrode, the guide apparatus having an implantation section constructed to at least partially enter a target object, wherein the implantation section has a first portion at a front end, a second portion adjoining a rear of the first portion, and a transition portion for connecting the first portion and the second portion, wherein the first portion is constructed to be able to pass through the engagement part, and the second portion is constructed to be unable to pass through the engagement part, so that the engagement part of the wire electrode can be stopped at the transition portion.

16. The guide apparatus of claim 15, wherein, when the engagement part of the wire electrode is stopped at the transition portion, a first section of the wire electrode that is adjacent to the engagement part is attached to the transition portion.

17. The guide apparatus of claim 15, wherein the transition portion is constructed to have a plane substantially perpendicular to a longitudinal direction of the guide apparatus, the plane being sized to be unable to pass through the engagement part, so that the engagement part of the wire electrode can be stopped at the plane of the transition portion.

18. The guide apparatus of claim 15, wherein the transition portion is constructed to have an increasing outer diameter in a direction from the first portion to the second portion, so that an outer peripheral edge at a specific position of the transition portion applies pressure to the engagement part of the wire electrode in a longitudinal direction of the guide apparatus, thus the engagement part of the wire electrode can be stopped at the specific position of the transition portion.

19. The guide apparatus of claim 15, wherein the guide apparatus further has a fixation section for fixing the guide apparatus to a driving apparatus for moving the guide apparatus, the fixation section being located at a rear end of the guide apparatus.

20. The guide apparatus of claim 19, wherein the fixation section and the implantation section are constructed integrally.

21. The guide apparatus of claim 19, wherein the fixation section is constructed to be hollow, and the second portion of the implantation section can be received and fixed in the hollow structure of the fixation section.

22. The guide apparatus of claim 21, wherein a stiffness of a material of the fixation section is higher than a stiffness of a material of the implantation section.

23. The guide apparatus of claim 15, wherein the engagement part is constructed with a through hole, and a size of the first portion is smaller than a size of the through hole, and a size of the second portion is larger than the size of the through hole.

24. The guide apparatus of claim 15, wherein at least the implantation section is made of a metal, alloy, carbon fiber or diamond with a Young's modulus greater than 20 GPa.

25. The guide apparatus of claim 15, wherein at least the implantation section is made of tungsten or stainless steel by etching.

26. The guide apparatus of claim 15, wherein the first portion is constructed to be columnar with a diameter between 5 μm and 15 μm, and/or, the second portion is constructed to be a columnar with a diameter between 40 μm and 70 μm.

27. The guide apparatus of claim 19, wherein the fixation section is constructed to be a columnar with a diameter between 100 μm and 200 μm.

28. A method for manufacturing a guide apparatus, comprising:
immersing a first section of an etchable blank into an etchant, and performing a first electrochemical etching on the first section, so as to obtain a semi-finished product with one step structure; and
immersing a first sub-section at an end of the first section into the etchant, and performing a second electrochemical etching on the first sub-section, so as to obtain the guide apparatus with two step structures,
wherein an implantation section of the guide apparatus is obtained on the first section, the implantation section having a first portion corresponding to the first sub-section and a second portion corresponding to the rest of the first section except for the first sub-section, the first portion being thinner than the second portion.

29. The method of claim 28, wherein the etchant is an alkaline solution.

30. The method of claim 28, wherein the etchant is a potassium hydroxide solution.

31. The method of claim 28, wherein the immersing a first section of an etchable blank into an etchant comprises:
laterally immersing the first section of the blank into the etchant, so that an electrochemical etching state of the first section can be observed from above with an observation apparatus for observing the electrochemical etching state of the first section.

32. The method of claim 31, wherein the observation apparatus observes the electrochemical etching state of the first section vertically from above.

33. The method of claim 31, wherein the observation apparatus is constructed as an optical microscope.

34. The method of claim 28, further comprising: covering at least a second sub-section of the first section that adjoins the first sub-section with an isolation material before performing the second electrochemical etching on the first sub-section of the first section, so that the second sub-section is not in contact with the etchant during the second electrochemical etching.

35. The method of claim 34, wherein the isolation material is a silicone adhesive.

36. The method of claim 34, wherein the covering a second sub-section with an isolation material comprises: making an end of the isolation material that is close to the first sub-section form a substantially flat surface which has an angle with respect to a longitudinal direction of the second sub-section that is not less than a threshold angle.

37. The method of claim 36, further comprising: placing the blank upright and positioning the first sub-section above the second sub-section, and pouring flowable isolation material into a container that is provided around the outside of the second sub-section and has an upward opening, so that an upper surface of the poured isolation material forms the substantially flat surface under an effect of gravity.

38. The method of claim 28, further comprising:
performing electrochemical etching on multiple blanks simultaneously, and separately controlling the electrochemical etching process of each blank respectively.

39. The method of claim 38, wherein, before performing the first electrochemical etching, each blank is connected in series with a separate switching element respectively, and corresponding sub-circuits each including a corresponding blank and a corresponding switching element are connected in parallel to a main circuit.

40. The method of claim 28, wherein an etching speed of the second electrochemical etching is less than an etching speed of the first electrochemical etching.

41. A method for manufacturing a guide apparatus, comprising:
immersing a first sub-section at an end of a first section of an etchable blank into an etchant, and performing a first electrochemical etching on the first sub-section, so as to obtain a semi-finished product with one step structure; and
immersing the first section into the etchant, and performing a second electrochemical etching on the first section, so as to obtain the guide apparatus with two step structures,
wherein an implantation section of the guide apparatus is obtained on the first section, the implantation section having a first portion corresponding to the first sub-section and a second portion corresponding to the rest of the first section except for the first sub-section, the first portion being thinner than the second portion.

42. A method for guiding a wire electrode, comprising:
placing, at a first position, the wire electrode with a through hole provided at an end portion thereof;
aligning a guide apparatus with the through hole, wherein the guide apparatus comprises, in a length direction thereof, a first portion at a front end and a second portion connected to the first portion, a maximum outer diameter size of the first portion being smaller than a size of the through hole and a minimum outer diameter size of the second portion being larger than the size of the through hole, wherein the second portion and the first portion are connected via a step structure;
moving the guide apparatus forward so that the first portion passes through the through hole and the end portion of the wire electrode is stopped at the step structure due to pressure from the step structure in a longitudinal direction of the guide apparatus; and
moving the guide apparatus to guide at least the end portion of the wire electrode from the first position to a second position.

43. The method of claim 42, wherein the wire electrode is placed at the first position by adhering the wire electrode to a substrate located at the first position, and the method further comprises:
at least partially peeling off the wire electrode from the substrate by moving the guide apparatus, so as to guide at least the end portion of the wire electrode from the first position to the second position.

44. A method for using a guide apparatus, comprising:
bringing the guide apparatus close to an engagement part of a wire electrode, wherein the guide apparatus comprises a thinner first portion at a front end, a thicker second portion adjoining a rear of the first portion, and a transition portion connecting the first portion and the second portion, wherein the first portion is constructed to be able to pass through the engagement part, and the second portion is constructed to be unable to pass through the engagement part;
passing the first portion through the engagement part of the wire electrode, and stopping the engagement part of the wire electrode at the transition portion; and
guiding the wire electrode using the guide apparatus.

45. The method of claim 44, further comprising:
spraying a liquid on the wire electrode before guiding the wire electrode using the guide apparatus, so that at least a first section of the wire electrode is attached to the transition portion and/or the second portion of the guide apparatus.

46. The method of claim 44, wherein the engagement part is constructed with a through hole, and an outer diameter of the first portion is smaller than an inner diameter of the through hole, and an outer diameter of the second portion is larger than the inner diameter of the through hole.

47. A method for guiding a wire electrode using a guide apparatus, the guide apparatus comprising a thinner first portion at a front end, a thicker second portion adjoining a rear of the first portion, and a transition portion for connecting the first portion and the second portion, wherein the first portion is constructed to be able to pass through an engagement part, the second portion is constructed to be unable to pass through the engagement part, a first end portion of the wire electrode is constructed with the engagement part, and the method comprises:
passing the first portion through the engagement part, and stopping the engagement part of the wire electrode at the transition portion;
further moving the guide apparatus to apply pull force to the first end portion of the wire electrode, so as to at least partially separate the wire electrode from a fixation apparatus for the wire electrode;
guiding the wire electrode to a target position using the guide apparatus.

48. The method of claim 47, wherein the further moving the guide apparatus to apply pull force to the first end portion of the wire electrode comprises:
moving the guide apparatus towards the target position at an angle with respect to a fixation plane for the wire electrode on the fixation apparatus to apply pull force to the first end portion of the wire electrode.

49. The method of claim 47, wherein the engagement part is constructed as a through hole, and an outer diameter of the first portion is smaller than an inner diameter of the through hole, and an outer diameter of the second portion is larger than the inner diameter of the through hole.
